(19) United States
(12) Patent Application Publication
(10) Pub. No.: US 2024/0407817 A1
(43) Pub. Date: Dec. 12, 2024

(54) DEVICE AND METHOD FOR DISPENSING A LIQUID STARTING COMPONENT OF A BONE CEMENT PASTE

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/680,672

(22) Filed: May 31, 2024

(30) Foreign Application Priority Data
Jun. 6, 2023 (EP) 23177507.3

(51) Int. Cl. A61B 17/88

(52) U.S. Cl. CPC A61B 17/8805

(57) ABSTRACT

Device for dispensing a liquid starting component of a bone cement paste from a glass ampoule. comprising a housing, a holder arranged in the housing for receiving a glass ampoule, and an outlet for dispensing the liquid starting component from the housing.

### CROSS-REFERENCE TO RELATED APPLICATIONS

This application claims priority pursuant to 35 U.S.C. 119(a) to European Application No. 23177507.3, filed Jun. 6, 2023, which application is incorporated herein by reference in its entirety.

### FIELD OF THE INVENTION

The invention relates to a device for dispensing a liquid starting component of a bone cement paste from a glass ampoule, comprising a housing, a holder arranged in the housing for receiving a glass ampoule, and an outlet for dispensing the liquid starting component from the housing. The invention further relates to a mixing device comprising such a device and a mixing unit, as well as a method for dispensing a liquid starting component of a bone cement paste by means of such a device.

### BACKGROUND OF THE INVENTION

Considerable efforts are being made to demonstrate devices and methods for providing bone cement by means of which bone cement paste can be provided simply, reliably, and quickly. An important aspect in the provision of bone cement paste is avoiding air inclusions, such as gas bubbles, in the bone cement. To avoid these, a plurality of vacuum cementing systems have been described, of which the following are mentioned by way of example: U.S. Pat. Nos. 6,033,105 A, 5,624,184 A, 4,671,263 A, 4,973,168 A, 5,100,241 A, WO 99/67015 A1, EP 1020167 A2, U.S. Pat. No. 5,586,821 A, EP 1016452 A2, DE 3640279 A1, WO 94/26403 A1, EP 1005901 A2, EP 1886647 A1, U.S. Pat. No. 5,344,232 A.

There is a desire in the market to simplify the provision of bone cement paste. A further development is the development of cementing systems in which both starting components are stored in separate areas of the mixing systems and are only mixed together in the cementing system immediately before the cementing application. Such closed, so-called full-prepacked systems are named for example in the following documents: EP 0 692 229 A1, DE 10 2009 031 178 B3, U.S. Pat. Nos. 5,997,544 A, 6,709,149 B1, DE 698 12 726 T2, EP 0 796 653 A2, U.S. Pat. No. 5,588,745 A.

In the mentioned full-prepacked systems, a monomer liquid is mixed with a bone cement powder by mechanical mixing, for example using a mixing rod.

A particular challenge in the mentioned systems is the provision of the liquid starting component, especially the monomer liquid for a PMMA bone cement paste. This liquid starting component is often stored in glass ampoules, which offers advantages in particular due to the case with which such glass ampoules can be sterilized.

In order to remove the liquid starting component from a commercially standard glass ampoule, however, the ampoule head has to be detached from the ampoule body of the glass ampoule and removed. A series of patent specifications have been made known for opening glass ampoules in closed devices. Devices for opening ampoules have been disclosed e.g. in documents DE19532015A1, WO9718031A1, and WO2010012114A1.

EP2404864B1 discloses a device in which an ampoule is arranged in an ampoule holder and wherein the ampoule holder is elastically deformable in the region of the ampoule neck. The ampoule holder is not deformable in the region of the ampoule head and the ampoule body. By bending the ampoule holder over the elastically deformable portion of the ampoule holder, which has a hinge function, the ampoule head can be pressed against the non-deformable wall of the ampoule holder. The ampoule head can thereby break off and fall onto a screen. The monomer liquid can consequently flow out of the opened ampoule through the screen. For the device described there to function, it is advantageous that it can be connected with mechanical stability to a mixing system to be supplied with monomer liquid, because a considerable tensile load can occur at the attachment to the mixing system during the bending process.

There is a desire on the market for further devices which allow simple and safe opening of glass ampoules for releasing liquid starting components for bone cement paste.

### OBJECTS

One object of the present invention is to overcome, at least in part, one or more of the disadvantages resulting from the prior art.

In particular, the invention is based on the aim of providing a device which allows simple, rapid, and safe, in particular injury-free, opening of one or more glass ampoules comprising a liquid starting component. In particular, the glass ampoule or glass ampoules should be opened with as little effort as possible while avoiding the use of additional, separate tools. Furthermore, the opening of the glass ampoule with as few components as possible is to be enabled. The liquid starting component should also be made available as quickly and without loss as possible to mix a bone cement paste. The conveying of the monomer liquid out of the device in order to provide the bone cement paste should be capable of being carried out with the smallest possible effort. The device should make it possible to open a plurality of, in particular two, glass ampoules simultaneously or separately, and with a time offset, so as to conduct fluid.

The device should be capable of being operated in as few work steps as possible in order to minimize sources of user error.

The device can be used in combination with a mixing unit in order to enable provision of a bone cement paste.

Furthermore, the device is to be able to provide a liquid starting component in a sterile and

contamination-free manner. A user of the device should preferably not come into contact with the glass ampoule to be opened.

It is a further object of the invention to provide a mixing device by means of which at least some of the objects already described are at least partially achieved.

It is a further object of the invention to provide a method for dispensing a liquid starting

component of a bone cement paste by means of which at least some of the objects already described are at least partially achieved.

### PREFERRED EMBODIMENTS OF THE INVENTION

A contribution to the at least partial fulfillment of at least one of the aforementioned objects is made by the features of the independent claims. The dependent claims provide preferred embodiments that contribute to at least partial fulfillment of at least one of the objects.

A first embodiment of the invention is a device for dispensing a liquid starting component of a bone cement paste from a glass ampoule, comprising a housing, a holder arranged in the housing for receiving a glass ampoule,
an outlet for dispensing the liquid starting component from the housing, characterized in
that the housing has a deformable region which is designed and arranged to be displaceable by pressure against the glass ampoule received in the holder and to open the glass ampoule in a fluid-conducting manner, in particular in such a way that the liquid starting component can flow out into the device, in particular into the housing.

In one embodiment of the device, the deformable region of the housing has, on the side facing the holder, i.e. inside the housing, a substantially dimensionally stable pressure piece which can be displaced by pressure against a glass ampoule accommodated in the holder, in particular filled with a liquid starting component of a bone cement paste, in particular in order to open it in a fluid-conducting manner by this exertion of pressure. This embodiment is a second embodiment of the invention, which is preferably dependent upon the first embodiment of the invention.

In one embodiment of the device, the deformable region has a partial region, in particular a circumferential partial region, having a thinner wall thickness than the rest of the housing. This embodiment is a third embodiment of the invention, which is preferably dependent upon the first or second embodiment of the invention.

In one embodiment of the device, the deformable region is formed at least in portions from an elastic material, in particular in order to allow the deformable region to return substantially completely to its initial position after a glass ampoule has been opened in a fluid-conducting manner. This embodiment is a fourth embodiment of the invention, which preferably depends upon one of the preceding embodiments of the invention.

In one embodiment of the device, the deformable region is arranged between the holder and the outlet. This embodiment is a fifth embodiment of the invention, which preferably depends upon one of the preceding embodiments of the invention.

In one embodiment of the device, the holder is configured to store a glass ampoule in the housing in such a way that an ampoule head of the glass ampoule adjoins the deformable region of the housing. This embodiment is a sixth embodiment of the invention, which preferably depends upon one of the preceding embodiments of the invention.

In one embodiment of the device, between the holder and the outlet a filter element is arranged that is designed to retain, in the device, fragments of a glass ampoule opened in a fluid-conducting manner in the device. This embodiment is a seventh embodiment of the invention, which preferably depends upon one of the preceding embodiments of the invention.

In one embodiment of the device, the filter element is arranged between the deformable region and the outlet. This embodiment is an eighth embodiment of the invention, which is preferably dependent on the seventh embodiment of the invention.

In one embodiment of the device, a collection region is arranged between the holder and the outlet in order to receive a liquid starting component from a glass ampoule opened a fluid-conducting manner in the device. This embodiment is a ninth embodiment of the invention, which preferably depends upon one of the preceding embodiments of the invention.

In one embodiment of the device, the device has a conveying means for conveying a liquid starting component, in particular a liquid starting component of a bone cement paste, from the collection region through the outlet out of the device. This embodiment is a tenth embodiment of the device, which is preferably dependent on the ninth embodiment of the invention.

In one embodiment of the device, the conveying means is a rubber balloon. This embodiment is an eleventh embodiment of the invention, which is preferably dependent on the tenth embodiment of the invention.

In one embodiment of the device, the conveying means is a piston. This embodiment is a twelfth embodiment of the invention, which is preferably dependent on the tenth embodiment of the invention.

A thirteenth embodiment of the invention is a mixing device for providing a bone cement paste from two starting components, comprising a device according to any one of the first to twelfth embodiments of the invention and a mixing unit, wherein the mixing unit is designed and configured to mix, in particular to mix and thus provide, the bone cement paste from the two starting components.

In one embodiment of the mixing device, the device contains a glass ampoule comprising a liquid starting component as the first starting component of the bone cement paste, and the mixing unit contains a bone cement powder as the second starting component of the bone cement paste. This embodiment is a fourteenth embodiment of the invention, which is preferably dependent upon the thirteenth embodiment of the invention.

A fifteenth embodiment of the invention is a method for dispensing a liquid starting component of a bone cement paste from a glass ampoule by means of a device according to any one of the first to twelfth embodiments of the invention, comprising the following steps:
a. providing a glass ampoule containing the liquid starting component in the holder;
b. opening the glass ampoule in a fluid-conducting manner by exertion of pressure on the deformable region so that this region is pressed against the glass ampoule;
c. conveying the liquid starting component through the outlet out of the device.

### GENERAL

In the present description, specifications of ranges also contain the values specified as limits. A specification of the type “in the range from X to Y” with respect to a quantity A consequently means that A can take the values X, Y and values between X and Y. One-sidedly limited ranges of the type “up to Y” for a size A accordingly mean as a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastics” comprises a plastics content of ≥95 to ≤100% by weight. For example, a “substantially complete filling of a volume B” comprises a filling of ≥95 to ≤100% by volume of the total volume of B.

Terms such as “proximal” and “distal” merely serve to designate the spatially opposite ends of the device or other structural units of the device, and do not allow any conclusions to be drawn about the orientation in relation to a human body-for example, a user of the device. “Distal to . . . ” and “proximal to . . . ” or similar formulations merely express the spatial arrangement of two structural units of the device in relation to one another.

### DETAILED DESCRIPTION

A first subject matter of the invention relates to a device for dispensing a liquid starting component of a bone cement paste from a glass ampoule, comprising a housing, a holder arranged in the housing for receiving a glass ampoule, an outlet for dispensing the liquid starting component from the housing, characterized in that the housing has a deformable region which is designed and arranged to be displaceable by pressure against the glass ampoule received in the holder and to open the glass ampoule in a fluid-conducting manner, in particular in such a way that the liquid starting component can flow out into the device, in particular into the housing.

The device is used in particular for fluid-conducting opening of one or more glass ampoules provided in the device. For this purpose, the housing of the device, in particular a wall of the housing, has a deformable region which can be displaced into an interior of the housing and thus pressed against a glass ampoule mounted there. The deformable region can thus be used from outside the device, in particular by a user of the device, to exert such high pressure on the glass ampoule stored in the device that it breaks open at least in portions, and a liquid starting component stored in the glass ampoule can flow out into the housing. This allows simple and rapid fluid-conducting opening of a glass ampoule without additional auxiliary elements. In addition, such opening without direct contact with the glass ampoule reduces the risk of injury to the user of the bone cement paste to be produced, especially when opening the glass ampoule in a fluid-conducting manner. The device is preferably designed to open the glass ampoule in a fluid-conducting manner under sterile conditions and to provide it for mixing the bone cement paste. The housing is preferably designed for sterile storage of a glass ampoule. For this purpose, the housing is preferably substantially closed off from the surroundings of the device, preferably hermetically.

The housing of the device is used to receive and store at least one glass ampoule. Preferably, a plurality of, preferably two, glass ampoules can be stored in the housing, preferably next to one another, in particular so as to be sterile, safe for transport, and preferably hermetically separated from the exterior world.

A holder is arranged within the housing in order to receive at least one, preferably two, glass ampoules. Preferably, the holder encloses the at least one glass ampoule at least in portions, so that it can be stored in the device safely for transport. The holder also serves to fix the at least one glass ampoule so that it can be opened in a fluid-conducting manner by means of the deformable region through the exertion of pressure.

In order to provide a liquid starting component from a glass ampoule for mixing a bone cement paste, the device has a fluid-conducting outlet, such as a feedthrough, e.g. a tube or a pipe. After the glass ampoule is opened in a fluid-conducting manner, the liquid starting component can be provided from the device through the outlet to a desired location, in particular for mixing a bone cement paste. The outlet can be arranged at different locations on the device, wherein it is preferred to arrange it at a distal end, in particular at a distal end of the longitudinal axis of the device.

In order to enable a glass ampoule stored in the holder to be opened in a fluid-conducting manner, the deformable region can be designed in different ways, as long as the deformable region is made so that it can be inserted into the interior of the housing in such a way that, via said region, pressure can be exerted on the glass ampoule from outside the device in order to open the glass ampoule in a fluid-conducting manner.

For example, the deformable region can be formed completely from a deformable material.

In a preferred embodiment, more than one glass ampoule can be accommodated in the holder in the device, and the device has a separate deformable region for each of these glass ampoules that can be accommodated, so that in each case a deformable region can open precisely one glass ampoule in a fluid-conducting manner. This allows glass ampoules to be opened in a fluid-conducting manner as needed. For example, in one embodiment, the device has a holder for two glass ampoules and two separate deformable regions. The two deformable regions are designed such that each of the deformable regions can open one of the two glass ampoules in a fluid-conducting manner.

One embodiment of the device is characterized in that the deformable region of the housing has, at least on the side facing the holder, i.e. on the inside of the housing, a substantially dimensionally stable pressure piece which can be displaced by pressure, in particular together with the deformable region, against a glass ampoule held in the holder in order to open it in a fluid-conducting manner. The pressure piece is substantially dimensionally stable, i.e. is substantially not deformable by the user during regular use of the device, which facilitates fluid-conducting opening of a glass ampoule arranged in the holder by exerting pressure on the deformable region. For this purpose, the pressure piece is arranged so that it comes into direct contact with the glass ampoule to be opened at the latest when pressure is exerted on the deformable region. Furthermore, a user of the device is better protected by the pressure piece against possible glass fragments which can result during the fluid-conducting opening of a glass ampoule. In one variant, the pressure piece is fastened at least to the side of a deformable material facing the holder, for example glued or positively and/or non-positively fastened in an enclosure, in order to form a deformable region according to this embodiment. In a further variant, the deformable region consists centrally of the pressure piece, which is surrounded all around by a deformable material in order to form a deformable region according to this embodiment.

The deformable region can be made from a material that is easier to deform than the rest of the housing, or from the same material as the rest of the housing.

One embodiment of the device is characterized in that the deformable region has a partial region, in particular a circumferential partial region, having a thinner wall thickness than the rest of the housing. This allows the housing to be manufactured from a uniform material, the deformable region being easier to shape than the rest of the housing. In one variant, the rest of the housing has a wall thickness that, although the material itself is deformable, is substantially non-deformable for a user of the device. In one variant, the deformable region has a thinner wall thickness in the circumferential partial region, i.e. on the outer partial region which adjoins the rest of the housing, than in its central partial region. The central partial region can be designed as a substantially dimensionally stable pressure piece.

Preferably, the housing together with the deformable region comprises a rubber-elastic material with a Shore hardness A greater than 60; in particular, the housing together with the deformable region is made of such a material. Examples of such materials are biocompatible elastomers such as silicone or EPDM.

One embodiment of the device is characterized in that the deformable region on the circumferential partial region has a fold-like, in particular accordion-like, structure which allows insertion in the direction of the holder. There is an at least partial unfolding of these folds here.

One embodiment of the device is characterized in that the deformable region is formed from an clastic material at least in portions, preferably at least on the circumferential partial region. This allows the deformable region to be automatically reset into its starting position, or at least in the direction toward its starting position, after a glass ampoule mounted in the holder has been opened in a fluid-conducting manner. It is thus ensured that the deformable region does not remain in the interior of the housing in a disturbing manner during the further use of the device, in particular during the flowing out or conveying of the liquid starting component stored in the glass ampoule.

The deformable region can be arranged at different locations along the longitudinal axis of the housing.

One embodiment of the device is characterized in that the deformable region is arranged spatially between the holder and the outlet. A glass ampoule mounted in the holder can thus be opened in a fluid-conducting manner in spatial proximity to the outlet without the liquid starting component having to flow through or past the holder in the direction of the outlet. This facilitates the substantially complete conveying of the liquid starting component out of the device.

The device according to the invention is designed in particular for glass ampoules which have an ampoule body, an ampoule head, and an ampoule neck which structurally connects the ampoule body and the ampoule head to each other. The ampoule body represents a large part of the glass ampoule and serves to receive at least a large portion of the liquid starting component in its interior. The ampoule neck has a reduced outer diameter compared to the ampoule body and serves as a predetermined breaking point for breaking off the ampoule head, which is small compared to the ampoule body. Such glass ampoules are well known to those skilled in the art.

Preferably, the holder is designed to accommodate an ampoule neck of a glass ampoule. For example, for this purpose the holder is formed as a shoulder, in particular an annular shoulder, wherein the ampoule neck is arranged in a feedthrough of the holder, the ampoule body rests on one side of the shoulder, and the ampoule head protrudes from the holder on the other side.

One embodiment of the device is characterized in that the holder is designed to store a glass ampoule in the housing in such a way that an ampoule head of the glass ampoule adjoins the deformable region of the housing. In this way, by exerting pressure from outside, the deformable region can be pressed directly onto the ampoule head, which breaks off easily due to the predetermined breaking point of the glass ampoule, i.e. the ampoule neck, thus allowing the liquid starting component stored in the glass ampoule to flow out. This embodiment of the device facilitates the fluid-conducting opening of such a glass ampoule for a user.

One embodiment of the device is characterized in that between the holder and the outlet a filter element is arranged that is designed, in particular designed and arranged, to retain, in the device, fragments of a glass ampoule opened in fluid-conducting manner in the device. The filter element thus prevents the bone cement paste to be mixed from being contaminated by fragments of a glass ampoule.

To achieve this, a glass ampoule is preferably positioned proximally to the filter element, while the outlet is positioned distally to the filter element.

The filter element can for example comprise or consist of a screen, a porous material, or a membrane. Preferably, the filter element is a porous disc, for example made of sintered polypropylene particles, sintered or compressed polyethylene fibers, cellulose felt, or cardboard.

One embodiment of the device is characterized in that the filter element is arranged between the deformable region and the outlet. Fragments which can be produced by the fluid-conducting opening of a glass ampoule, such as a broken-off ampoule head, can thus be retained by the filter element inside the device and do not exit from the device through the outlet.

The device can be designed such that a liquid starting component can flow directly out of the device after a glass ampoule inside the device has been opened in a fluid-conducting manner. In order to achieve improved control over the time at which the liquid starting component flows out of the device, it can be useful to first leave the liquid starting component flowing out of the glass ampoule inside the device, and to only remove it from the device by active engagement.

To accomplish this, one embodiment of the device is characterized in that a collection region is arranged between the holder and the outlet in order to preferably completely receive a liquid starting component from one or more glass ampoules that are opened in a fluid-conducting manner in the device. The filter element can here be arranged proximally, i.e. in the direction of the glass ampoule, distally or also centrally within the collection region. Preferably, the collection region is designed in such a way that the ampoule head(s) of the glass ampoule or ampoules can also be accommodated in addition to the liquid starting component. The collection region is preferably dimensioned in such a way that an ampoule head can be mounted in freely rotatable fashion in the collection region, so that a liquid starting component that may be located in the ampoule head can flow out due to a rotation thereof. This facilitates substantially complete conveying of a liquid starting component out of the device.

Conveying of a liquid starting component out of the device, preferably from the collection region of the device, through the outlet can be initiated in different ways. For example, conveying under the effect of gravity alone can be enabled. For this purpose, it may be necessary to spatially align the device appropriately, for example with the outlet pointing towards the floor.

One embodiment of the device is characterized in that the device has a conveying means for conveying a liquid starting component out of the device, from the collection region through the outlet. The conveying means is used to actively trigger a conveying process of the liquid starting component by a user of the device.

One embodiment of the device is characterized in that the conveying means is a rubber balloon. A rubber balloon is, in the broadest sense, a bubble-like hollow body made of a deformable, in particular elastic, in particular reversibly elastic, material with a fluid-conducting opening. In order to function as a conveying means, the rubber balloon can be connected in fluid-conducting fashion to the interior of the housing via the fluid-conducting opening, so that a conveying pressure can be exerted on a liquid starting component stored in the interior of the housing by compressing the rubber balloon. The liquid starting component can be conveyed from the outlet of the device by the conveying pressure. To convey the liquid starting component, it may be necessary to compress the rubber balloon one or more times.

The rubber balloon is preferably located at an end of the device opposite the outlet and is connected there in a fluid-conducting manner to the interior of the housing.

One embodiment of the device is characterized in that the conveying means is a piston. In order to convey a liquid starting component out of the device, the piston interacts with a cylinder in such a way that insertion of the piston into the cylinder exerts a conveying pressure on a liquid starting component stored in the interior of the housing. The liquid starting component can be conveyed from the outlet of the device by the conveying pressure.

In one variant of this embodiment, the housing acts as a cylinder for the piston. In a further variant of this embodiment, the cylinder is connected in a fluid-conducting manner to the interior of the housing. Preferably, the cylinder is arranged at an end of the device opposite the outlet and is connected there in a fluid-conducting manner to the interior of the housing.

A further subject matter of the invention relates to a mixing device for providing a bone cement paste from two starting components, comprising
a device according to any of the preceding embodiments of the invention and a mixing unit, wherein the mixing unit is designed and arranged to mix the bone cement paste from the two starting components.

The mixing unit is used to mix a bone cement paste from a bone cement powder and the liquid starting component after conveying the liquid starting component into the mixing unit, in particular after conveying the liquid starting component into an interior of the mixing unit.

The mixing unit preferably has a hollow cylindrical cartridge. A hollow cylindrical cartridge is to be understood as a tubular container which has an interior space and a cartridge wall surrounding the interior space. The cross section of the cartridge can assume any desired shape. Due to the case of manufacture and the more reliable use of the device, the cross section, and preferably also the cross section of the interior space, is circular. This allows good handling for the user and, due to an absence of edges, reduces a risk of moving parts becoming wedged within the device. According to the invention, the cartridge can be made of a wide variety of materials or material combinations. For example, the device may be made of a polymer. The polymer is preferably a transparent polymer, since in this way the user can visually monitor the proper functioning of the device during use.

A dispensing piston is preferably arranged in the cartridge in order to apply the finally mixed bone cement paste from the mixing unit at a desired location. In order to mix the bone cement dough from the two starting components, the mixing unit preferably has a mixing element, such as a mixing rod, for example. Alternatively, a bone cement paste can also be mixed in the mixing unit by shaking it.

The device and the mixing unit are connected to each other in a fluid-conducting manner via the outlet of the device. In one variant, the device and the mixing unit are linearly connected to one another. In a further variant, the device and the mixing unit are connected to each other in a fluid-conducting manner via a fluid-conducting conduit, such as a tube or a pipe. In one variant, the outlet opens directly into the mixing unit.

The device and the mixing unit can be firmly connected or connectable to one another, for example in a positively and/or non-positively and/or materially connected manner. In one variant, the device and the mixing unit are made in one piece.

One embodiment of the mixing device is characterized in that the device contains a glass ampoule comprising a liquid starting component as the first starting component of the bone cement paste, and the mixing unit contains a bone cement powder as the second starting component of the bone cement paste. This allows the bone cement paste to be prepared in as sterile a manner as possible, since any transfer filling steps of the starting components by a user of the mixing device are eliminated and the user already receives a mixing device pre-filled with the starting components in a sterile state.

A further subject matter of the invention relates to a method for dispensing a liquid starting component of a bone cement paste from a glass ampoule by means of a device according to any one of the preceding embodiments, preferably a device as a component of a mixing device according to any one of the preceding embodiments of the invention, comprising the following steps:
a. providing a glass ampoule containing the liquid starting component in the holder;
b. opening the glass ampoule in a fluid-conducting manner by exertion of pressure on the deformable region so that this region is pressed against the glass ampoule;
c. conveying the liquid starting component through the outlet out of the device.

The provision of the glass ampoule in step a. can be carried out by a user of the device directly before its use, or the user is already provided with the device fitted with the glass ampoule, for example by the manufacturer of the device.

In order to open the glass ampoule stored in the holder in a fluid-conducting manner, in a step b. the deformable region is pressed from outside the device, which region is thus moved into, or further into, the interior of the housing and finally pressed against the glass ampoule. If the application of force here exceeds a limit value, at least a partial bursting of the glass ampoule occurs, and preferably a breaking off of an ampoule head of the glass ampoule, as a result of which it is opened in a fluid-conducting manner and the liquid starting component can flow out into the interior of the housing. The deformable region is preferably designed in such a way that after the exertion of pressure has ended it is returned automatically at least in the direction of, preferably completely into, its starting position before step b.

After the fluid-conducting opening of the glass ampoule, the liquid starting component preferably flows into a collection region of the device and remains there until the user of the device conveys the liquid starting component out of the device through active intervention.

In a step c., the liquid starting component is conveyed through the outlet of the device. In a variant of step c., the conveying is triggered solely by gravity. In a preferred variant of step c., the conveying takes place through active intervention by the user of the device. For example, the user of the device actuates a conveying means such as a piston or a rubber balloon to convey the liquid starting component through the outlet of the device.

Preferably, the liquid starting component is conveyed in step c. into a mixing unit connected in a fluid-conducting manner to the device according to one of the preceding embodiments, in which the liquid starting component is mixed with a bone cement powder as the second starting component of the bone cement paste in an optional step d. The device and the mixing unit together form a mixing device. In an optional step e., the bone cement paste provided in the mixing unit can preferably be dispensed directly therefrom at a desired location. For this purpose, the mixing device preferably comprises a dispensing plunger which is mounted so as to be movable axially within the mixing unit in order to dispense the provided bone cement paste from the mixing device, in particular from the mixing unit. For example, the dispensing piston can be moved in the mixing unit by means of a dispensing aid, such as a dispensing gun, to dispense the bone cement paste.

The device is used to provide a liquid starting component for a bone cement paste. A bone cement paste is a substance which is suitable in the field of medical technology for creating a stable connection between artificial joints, such as hip and knee joints, and bone material. Curing turns bone cement paste into bone cement. These bone cements are preferably polymethylmethacrylate bone cements (PMMA bone cements). PMMA bone cements have been used in medical applications for a long time, and go back to the work of Sir Charnley (cf. Charnley, J., Anchorage of the femoral head test of the drawing of the femur, J. Bone Joint Surg. 1960 (42) 28-30.). PMMA bone cements can be produced from a bone cement powder as the first starting component and a liquid starting component, in particular a monomer liquid, as the second starting component. With a suitable composition, the two starting components can be stably stored separately from one another. When the two starting components are brought into contact, the swelling of the polymer components of the bone cement powder produces a plastically deformable bone cement paste. A polymerization of the monomer is initiated by radicals. As the polymerization of the monomer progresses, the viscosity of the bone cement paste increases until it cures completely.

A bone cement powder is a powder which comprises at least one particulate polymethyl methacrylate and/or a particulate polymethylmethacrylate copolymer. Examples of copolymers are styrene and/or methyl acrylate. In one embodiment, the bone cement powder can additionally comprise a hydrophilic additive which supports the distribution of the monomer liquid within the bone cement powder. In a further embodiment, the bone cement powder can additionally comprise an initiator which initiates the polymerization. In a further embodiment, the bone cement powder can additionally comprise a radiopaque. In yet a further embodiment, the bone cement powder can additionally comprise pharmaceutically active substances, for example antibiotics.

Preferably, the bone cement powder comprises, as the hydrophilic additive, at least one particulate polymethyl methacrylate and/or a particulate polymethyl methacrylate copolymer, an initiator and a radiopaque, or consists of these components. Further preferably, the bone cement powder comprises at least one particulate polymethyl methacrylate and/or a particulate polymethylmethacrylate copolymer, an initiator, a radiopaque, and a hydrophilic additive, or consists of these components. Most preferably, the bone cement powder comprises at least one particulate polymethyl methacrylate and/or a particulate polymethylmethacrylate copolymer, an initiator, a radiopaque, a hydrophilic additive, and an antibiotic, or consists of these components.

According to the invention, the particle size of the particulate polymethyl methacrylate and/or of the particulate polymethylmethacrylate copolymer of the bone cement powder can correspond to the sieve fraction of less than 150 μm, preferably less than 100 μm.

According to the invention, the hydrophilic additive can be particulate and/or fibrous. In a further embodiment, the hydrophilic additive can be sparingly soluble, preferably insoluble, in methyl methacrylate. In a further embodiment, the hydrophilic additive can have an absorption capacity of at least 0.6 g of methyl methacrylate per gram of hydrophilic additive. In a further embodiment, the hydrophilic additive can have a chemical substance having at least one OH group. It can preferably be provided that the hydrophilic additive has covalently bonded OH groups on its surface. Examples of such preferred hydrophilic additives can be additives selected from the group comprising cellulose, oxycellulose, starch, titanium dioxide, and silicon dioxide, wherein fumed silicon dioxide is particularly preferred. In one embodiment, the particle size of the hydrophilic additive can correspond to the sieve fraction of less than 100 μm, preferably less than 50 μm and most preferably less than 10 μm. The hydrophilic additive can be contained in an amount of 0.1 to 2.5 wt. % relative to the total weight of the bone cement powder.

According to the invention, the initiator can contain dibenzoyl peroxide or consist of dibenzoyl peroxide.

According to the invention, a radiopaque is understood to be a substance which makes it possible to make the bone cement visible on X-ray diagnostic images. Examples of radiopaques can include barium sulfate, zirconium dioxide, and calcium carbonate.

According to the invention, the pharmaceutically active substance can comprise one or more antibiotics and optionally added co-factors for the one or more antibiotics. Preferably, the pharmaceutically active substance consists of one or more antibiotics and optionally added co-factors for the one or more antibiotics. Examples of antibiotics include, inter alia, gentamicin, clindamycin, and vancomycin.

According to the invention, the monomer liquid can comprise the monomer methyl methacrylate or consist of methyl methacrylate. In one embodiment, the monomer liquid comprises, in addition to the monomer, an activator dissolved therein, such as N,N-dimethyl-p-toluidine, or consists of methyl methacrylate and N,N-dimethyl-p-toluidine.

The features disclosed for the device are also disclosed for the mixing device and the method, and vice versa.

### FIGURES

The invention is further illustrated by way of example below by means of figures. The invention is not limited to the figures.

The following are shown

FIG. 1 a schematic longitudinal section of an exemplary device for dispensing a liquid starting component of a bone cement paste from a glass ampoule,

FIG. 2 the device from FIG. 1 with a glass ampoule which is opened in a fluid-conducting manner,

FIG. 3 the device from FIGS. 1 and 2 during the dispensing of the liquid starting component,

FIG. 4 a schematic longitudinal section of a further exemplary device for dispensing a liquid starting component of a bone cement paste from a glass ampoule, comprising a conveying means in the form of a rubber balloon,

FIG. 5 the device from FIG. 4 with a glass ampoule opened in a fluid-conducting manner,

FIG. 6 the device from FIGS. 4 and 5 during the dispensing of the liquid starting component,

FIG. 7 a schematic longitudinal section of a further exemplary device for dispensing a liquid starting component of a bone cement paste from a glass ampoule, comprising a conveying means in the form of a piston,

FIG. 8 the device from FIG. 7 with a glass ampoule opened in a fluid-conducting manner,

FIG. 9 the device from FIGS. 7 and 8 during the dispensing of the liquid starting component,

FIG. 10 a schematic longitudinal section of an exemplary mixing device for providing a bone cement paste from two starting components, comprising the device from FIGS. 1 to 3 and a mixing unit,

FIG. 11 a schematic longitudinal section of a further exemplary mixing device for providing a bone cement paste from two starting components, comprising a device for dispensing a liquid starting component of a bone cement paste and a mixing unit,

FIG. 12 a schematic longitudinal section of a further exemplary mixing device for providing a bone cement paste from two starting components, comprising a device for dispensing a liquid starting component of a bone cement paste and a mixing unit, and

FIG. 13 a flow chart of a method.

### DESCRIPTION OF THE FIGURES

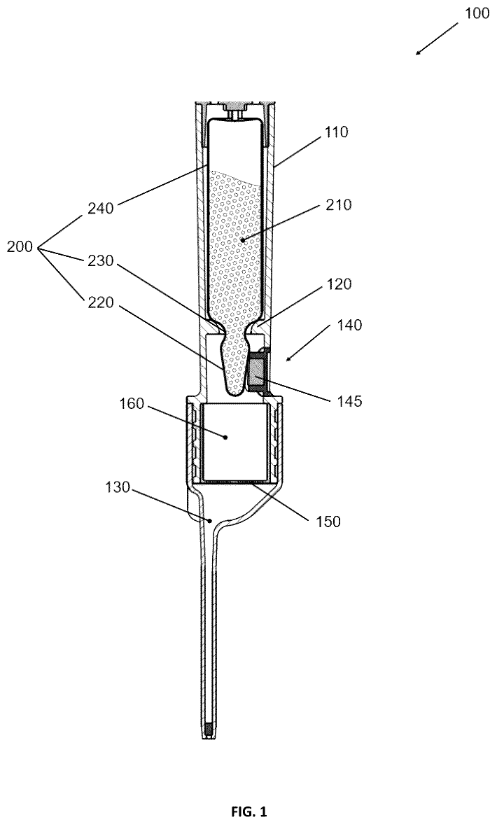

FIG. 1 shows a schematic longitudinal section of an exemplary embodiment of a device 100 for dispensing a liquid starting component 210 of a bone cement paste. The device 100 comprises a cylindrical housing 110 in which a glass ampoule 200 containing the liquid starting component 210 is stored. The glass ampoule 200 comprises an ampoule body 240, an ampoule head 220, and an ampoule neck 230 between the ampoule body 240 and ampoule head 220. The ampoule neck 230 is received in a holder 120 of the device 100 in order to fix the glass ampoule 200 substantially fixedly in the interior of the housing 110. In the embodiment shown, the holder 120 is formed as a shoulder, wherein the ampoule head 220 protrudes on one side of the holder 120 and the ampoule body 240 rests on the other side of the holder 120.

The glass ampoule 200 is accommodated by the holder 120 in the housing 110 in such a way that the ampoule head adjoins a deformable region 140 of the housing 110. The deformable region 140 is made of an elastic material and has a substantially dimensionally stable pressure piece 145 on the side facing the holder 120. The pressure piece 145 thus points in the direction of the ampoule head 220.

In order to enable dispensing of the liquid starting component 210 out of the device 100, the device has an outlet 130. In the embodiment of the device 100 shown, the outlet 130 is designed as a tube. A filter element 150 in the form of a porous disc is arranged inside the housing 110 between the outlet 130 and the holder 120, which disc can retain any fragments of the glass ampoule 200 in the device 100 after it has been opened. A collection region 160 for receiving the liquid starting component 210 is formed spatially between the filter element 150 and the holder 120.

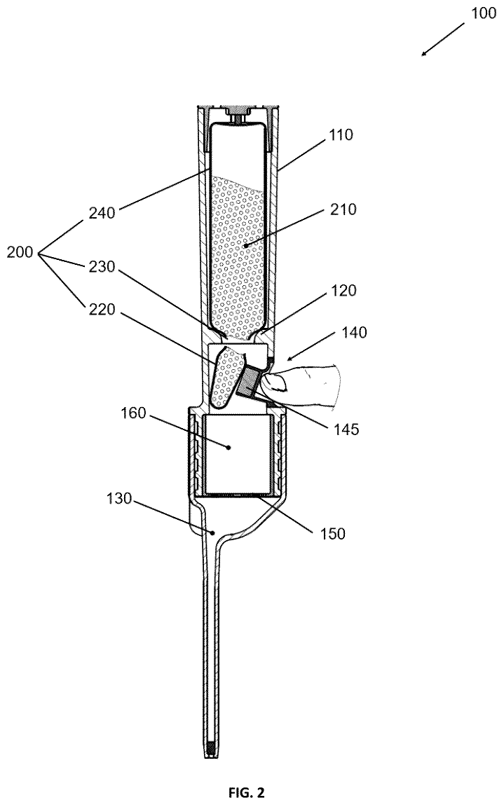

FIG. 2 shows the device 100 from FIG. 1 with a glass ampoule 200 opened in a fluid-conducting manner. To open the glass ampoule 200, the deformable region 140 was displaced from outside the device 100 in the direction of the holder 120, so that the pressure piece 145 was pressed against the ampoule head 220. This exertion of pressure exceeded the structural integrity of the ampoule neck 230, which acts as the predetermined breaking point of the glass ampoule 200, and the glass ampoule 200 was opened in a fluid-conducting manner by breaking off the ampoule head 220.

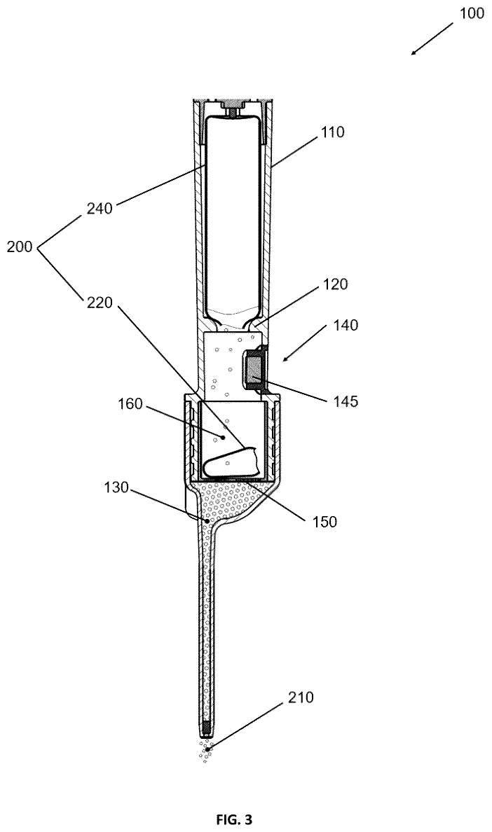

FIG. 3 shows the device 100 from FIGS. 1 and 2 during the dispensing of the liquid starting component 210. After the fluid-conducting opening of the glass ampoule 200 in FIG. 2, the ampoule head 220 fell into the collection region 160 due to gravity and was retained there by the filter element 150. The collection region 160 is dimensioned in such a way that the broken-off ampoule head 220 can be mounted in it in a freely rotatable manner, which facilitates the most complete possible outflow of any liquid starting component 210 that may remain in the ampoule head 220. The liquid starting component 210 has run out from the glass ampoule 200 which is open in a fluid-conducting manner, flowed through the collection region 160 and the filter element 150 under the force of gravity, and at least already partially exited from the device 100 through the outlet 130.

The external pressure exerted on the deformable region 140 in FIG. 2 has been removed, so that the deformable region 140 has substantially returned to its original starting position (sec FIG. 1) due to its material properties.

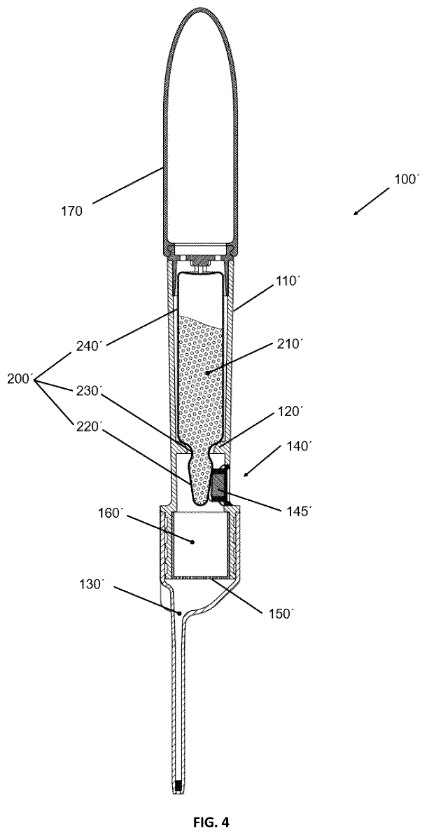

FIG. 4 shows a schematic longitudinal section of a further exemplary embodiment of a device 100′ for dispensing a liquid starting component 210′ of a bone cement paste. The embodiment of the device 100′ largely corresponds to the embodiment described above and shown in FIGS. 1 to 3, so that reference is made to the above description in order to avoid repetitions. Modifications of an embodiment shown in FIGS. 1 to 3 have the same reference sign with an apostrophe.

In contrast to the embodiment of FIGS. 1 to 3, the device 100′ has a conveying means 170 in the form of a rubber balloon. The conveying means 170 is arranged at an axial end of the housing 110′ opposite the outlet 130′ and is connected to the interior of the housing 110′ in a fluid-conducting manner.

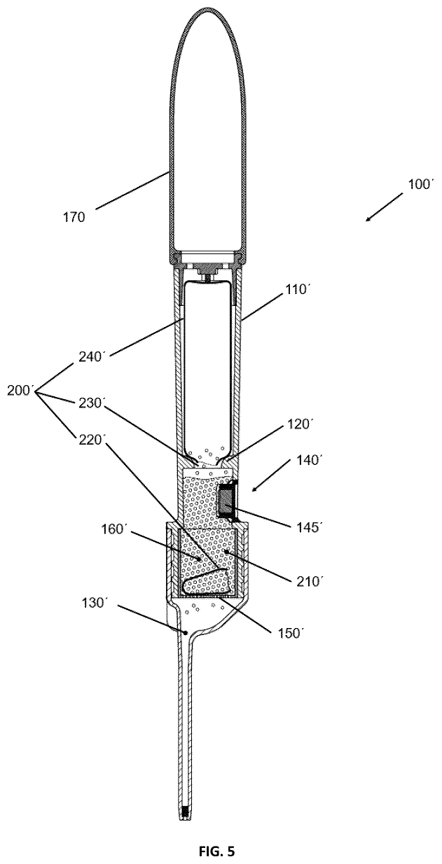

FIG. 5 shows the device 100 from FIG. 4 with a glass ampoule 200 opened in a fluid-conducting manner. To open the glass ampoule 200′, the deformable region 140′ was displaced from outside the device 100′ in the direction of the holder 120′, so that the pressure piece 145′ was pressed against the ampoule head 220′. As a result of this exertion of pressure, the structural integrity of the ampoule neck 230′, which functions as a predetermined breaking point of the glass ampoule 200′, was exceeded, and the glass ampoule 200′ was opened in a fluid-conducing manner by breaking off the ampoule head 220′. The liquid starting component 210′ has flowed out of the fluid-conducting opened glass ampoule 200′ into the collection region 160′ due to gravity, in which it remains for the time being without the intervention of a user of the device 100′. After the fluid-conducting opening of the glass ampoule 200′, the ampoule head 220′ fell into the collection region 160′ due to gravity and was retained there by the filter element 150′. The collection region 160′ is dimensioned in such a way that the broken-off ampoule head 220′ can be mounted in it in a freely rotatable manner, which facilitates the most complete possible outflow of any liquid starting component 210′ that may be in the ampoule head 220′.

After the fluid-conducting opening of the glass ampoule 200′, the deformable region 140′ has substantially returned to its original starting position (cf. FIG. 4) due to its material properties.

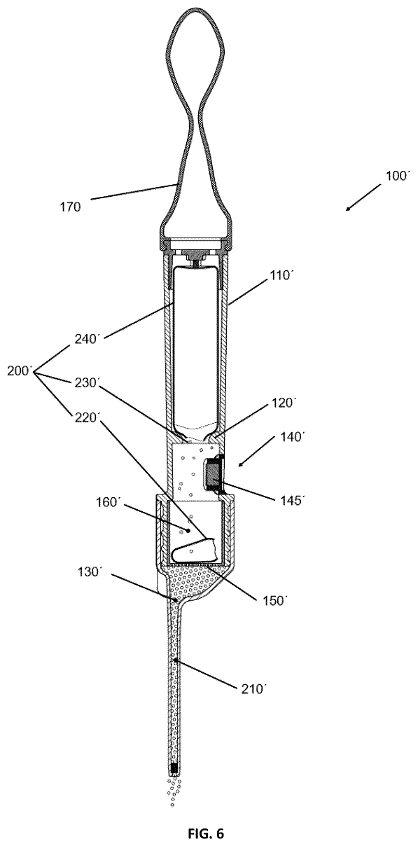

FIG. 6 shows the device 100′ from FIGS. 4 and 5 during dispensing of the liquid starting component 210′ through the outlet 130′. For this purpose, the conveying means 170 was compressed from outside the device 100′, which exerted a conveying pressure on the liquid starting component 210′ collected in the collection region 160′ (cf. FIG. 5) and caused it to be propelled through the outlet 130′. To completely dispense the liquid starting component 210′, it may be necessary to actuate the conveying means 170 several times.

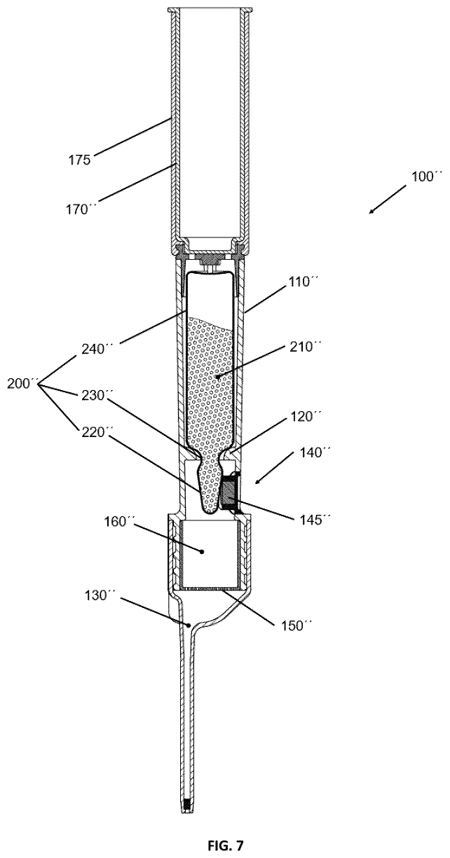

FIG. 7 shows a schematic longitudinal section of a further exemplary embodiment of a device 100″ for dispensing a liquid starting component 210″ of a bone cement paste. The embodiment of the device 100″ largely corresponds to the embodiments described above and shown in FIGS. 1 to 3 or 4 to 6, and therefore reference is made to the above descriptions in order to avoid repetitions. Modifications of an embodiment shown in FIGS. 1 to 3 or 4 to 6 have the same reference sign with two apostrophes.

In contrast to the embodiment of FIGS. 1 to 3, the device 100″ has a conveying means 170″ in the form of a piston. At an axial end of the housing 110″ opposite the outlet 130″, the conveying means 170 is arranged in a cylinder 175 of the housing, forming a piston-cylinder system, which is connected in a fluid-conducting manner to the interior of the housing 110′ containing the glass ampoule 220″.

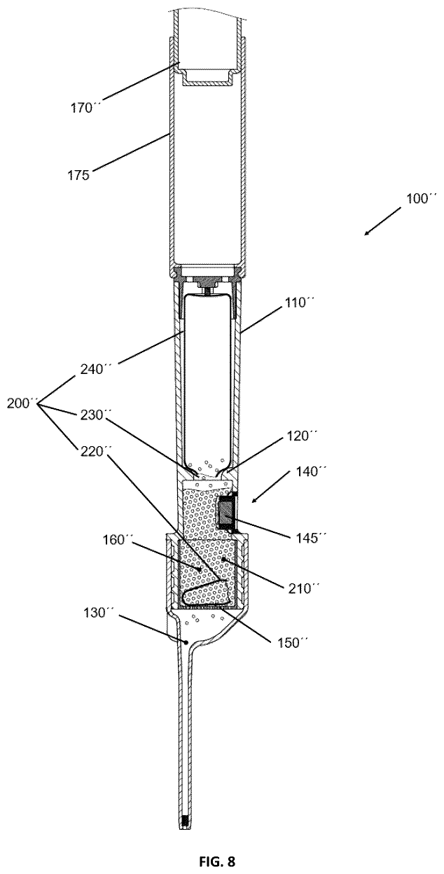

FIG. 8 shows the device 100″ from FIG. 7 with a glass ampoule 200 opened in a fluid-conducting manner. To open the glass ampoule 200′, the deformable region 140″ was displaced from outside the device 100″ in the direction of the holder 120″, so that the pressure piece 145″ was pressed against the ampoule head 220″. This exertion of pressure exceeded the structural integrity of the ampoule neck 230″, which acts as the predetermined breaking point of the glass ampoule 200″, and the glass ampoule 200″ was opened in a fluid-conducting manner by a breaking off of the ampoule head 220″. The liquid starting component 210″ has flowed out of the fluid-conducting opened glass ampoule 200″ into the collection region 160″ due to gravity, in which it remains for the time being without the intervention of a user of the device 100″. After the fluid-conducting opening of the glass ampoule 200″, the ampoule head 220″ fell into the collection region 160″ due to gravity and was retained there by the filter element 150″. The collection region 160″ is dimensioned in such a way that the broken-off ampoule head 220″ can be mounted in it in a freely rotatable manner, which facilitates the most complete possible outflow of any liquid starting component 210″ that may remain in the ampoule head 220″.

After the fluid-conducting opening of the glass ampoule 200″, the deformable region 140″ has substantially returned to its original starting position (cf. FIG. 7) due to its material properties.

In order to subsequently convey the liquid starting component 220″ from the collection region 160″ through the outlet 130″, the piston 170″ (only shown in portions) was partially pulled axially out of the cylinder 175.

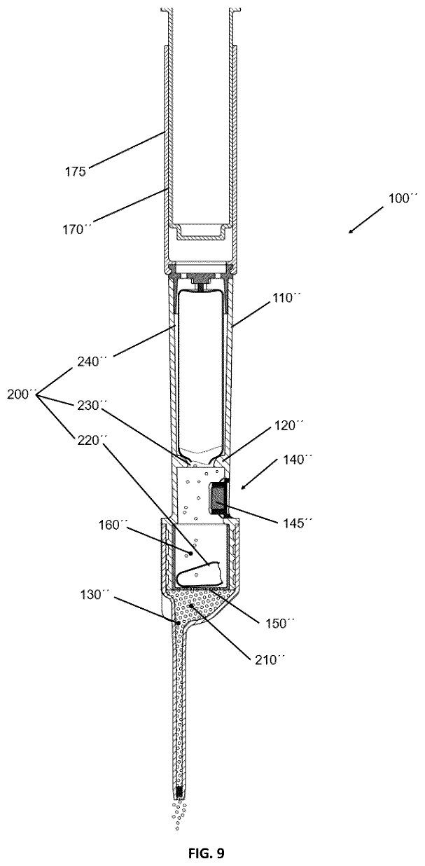

FIG. 9 shows the device 100″ from FIGS. 7 and 8 during dispensing of the liquid starting component 210″ through the outlet 130″. For this purpose, the conveying means 170 was pushed back into the cylinder 175, which exerted a conveying pressure on the liquid starting component 210″ collected in the collection region 160″ (cf. FIG. 8) and caused it to be propelled through the outlet 130″. To completely dispense the liquid starting component 210″, it may be necessary to actuate the conveying means 170″ several times.

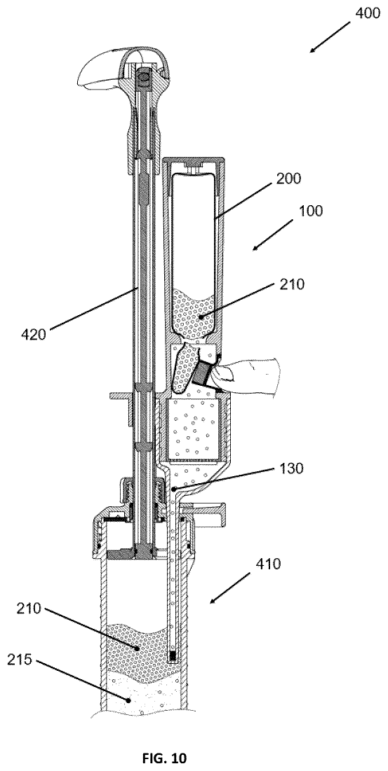

FIG. 10 shows a schematic longitudinal section of an exemplary mixing device 400 for providing a bone cement paste from two starting components, comprising the device 100 from FIGS. 1 to 3 and a mixing unit 410 (shown only in portions). The embodiment of the device 100 largely corresponds to the embodiment described above and shown in FIGS. 1 to 3, and therefore reference is made to the above description in order to avoid repetitions.

The device 100 is reversibly connected to the mixing unit 410 in a fluid-conducting manner via the tube-like outlet 130, wherein the liquid starting component 210 has already partially flowed into the mixing unit 410 after the glass ampoule 200 has been opened in a fluid-conducting manner. In an interior, the mixing unit 410 contains a bone cement powder 215 as the second starting component of the bone cement paste. In order to mix the bone cement paste from the starting components 210, 215, the mixing unit 410 has a mixing element 420 in the form of a mixing rod that can be moved axially and reversibly inside the mixing unit 410. After removal of the device 100, the mixing element 410 can provide the bone cement paste from the starting components 210, 215 by repeated axial displacement in the interior of the mixing unit 410.

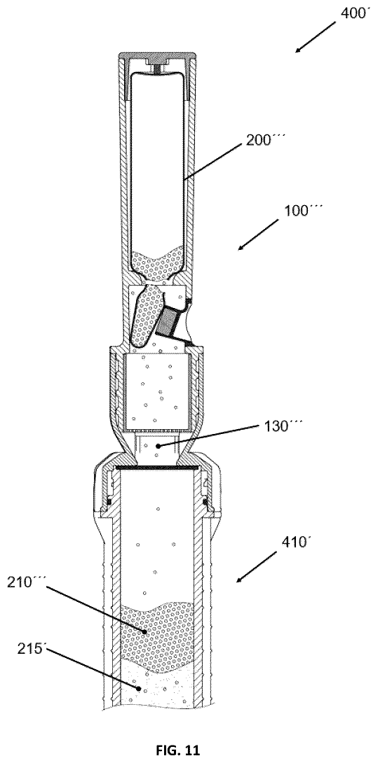

FIG. 11 shows a schematic longitudinal section of a further exemplary mixing device 400′ for providing a bone cement paste from two starting components, comprising a device 100″′ and a mixing unit 410′ (shown only in portions).

The embodiment of the device 100″′ largely corresponds to the embodiment described above and shown in FIGS. 1 to 3, and therefore reference is made to the above description in order to avoid repetitions. Modifications of an embodiment shown in FIGS. 1 to 3 have the same reference sign with three apostrophes.

The device 100″′ is reversibly connected to the interior of the mixing unit 410′ in a fluid-conducting manner in an axial line via the outlet 130″′, wherein the liquid starting component 210′″ has already partially flowed into the mixing unit 410′ after the glass ampoule 200′″ has been opened in a fluid-conducting manner. In an interior, the mixing unit 410′ contains a bone cement powder 215′ as the second starting component of the bone cement paste. In order to mix the bone cement paste from the starting components, the device 100″′ can be reversibly separated from the mixing unit 410′ and the two starting components 210″′, 215′ can be mixed with a mixing element (not shown).

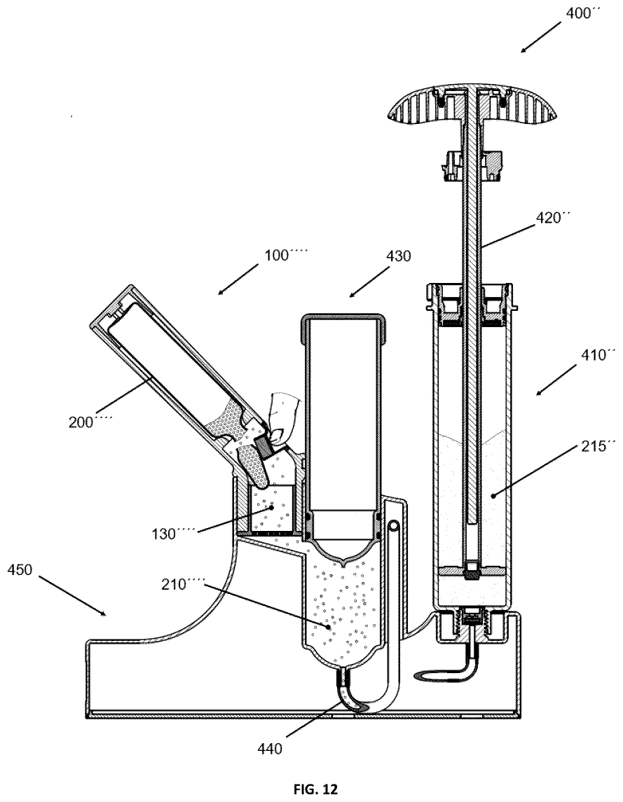

FIG. 12 shows a schematic longitudinal section of a further exemplary mixing device 400″ for providing a bone cement paste from two starting components, comprising a device 100″′ and a mixing unit 410″.

The embodiment of the device 100″″ largely corresponds to the embodiment described above and shown in FIGS. 1 to 3, and therefore reference is made to the above description in order to avoid repetitions. Modifications of an embodiment shown in FIGS. 1 to 3 have the same reference sign with four apostrophes.

The mixing device 410″ further comprises a stand element 450 via which the device 100″″ and the mixing unit 410″ are connected to one another in a fluid-conducting manner. After the glass ampoule 200″″ has been opened in a fluid-conducting manner, the liquid starting component 210″″ flows via the outlet 130″″ into a piston-cylinder system 430 of the stand element 450 and is collected there. Through actuation of the piston-cylinder system by the user of the mixing device 400″, the liquid starting component 210″″ can be conveyed via a hose 440 into the mixing unit 410, in particular into a bone cement powder 215″ stored there, in order to provide the bone cement paste through mixing by means of a mixing element 420″″ in the form of a mixing rod. For dispensing the bone cement paste thus provided, the mixing unit 410″ can be separated from the stand element 450.

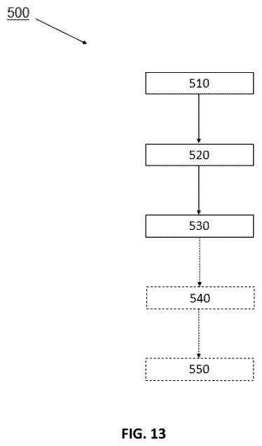

FIG. 13 shows a method 500 for dispensing a liquid starting component 210, 210′, 210″, 210′″, 210″″ of a bone cement paste from a glass ampoule 200, 200′, 200″, 200″′, 200″″ by means of a device 100, 100′, 100″, 100″′, 100″″ according to FIGS. 1 to 12, comprising the steps 510 to 530 and optionally the steps 540 and 550.

In a step 510, at least one glass ampoule 200, 200′, 200″, 200″′, 200″″ is provided in the holder 120, 120′, 120″ of the device 100, 100′, 100″, 100″′, 100″″. This can be carried out by a user of the device 100, 100′, 100″, 100″′, 100″″ or the user is provided with the device 100, 100′, 100″, 100″′, 100″″ with one or more prefilled glass ampoules 200, 200′, 200″, 200″′, 200″″.

In order to open the glass ampoule 200, 200′, 200″, 200″′, 200″″, which is mounted in the holder 120, 120′, 120″ in a fluid-conducting manner, in a step 520 pressing takes place from outside the device 100, 100′, 100″, 100′″, 100″″ on the deformable region 140, 140′, 140″, which is thus displaced into or further into the interior of the housing 110, 110′, 110″ and is finally pressed against the glass ampoule 200, 200′, 200″, 200″′, 200″″. If the force applied exceeds a limit value, the glass ampoule 200, 200′, 200″, 200″′, 200″″ at least partially bursts, and preferably an ampoule head 220, 220′, 220″ of the glass ampoule 200, 200′, 200″′, 200″′, 200″″ breaks off, whereby this head is opened in a fluid-conducting manner and the liquid starting component 210, 210′, 210″, 210″′, 210″″ can flow out into the interior of the housing 110, 110′, 110″. Preferably, the deformable region 140, 140′, 140″ is designed in such a way that, after the exertion of pressure has ended, the region is returned automatically at least in the direction of, preferably completely into, its starting position before step 520.

After the fluid-conducting opening of the glass ampoule 200, 200′, 200″, 200″′, 200″″, the liquid starting component 210, 210′, 210″, 210″′, 210″″ preferably flows into a collection region 160, 160′, 160″ of the device 100, 100′, 100″, 100″′, 100″″ and remains there until the user of the device 100, 100′, 100″, 100″′, 100″″ conveys the liquid starting component 210, 210′, 210″, 210′″, 210″″ out of the device 100, 100′, 100″, 100′′, 100″″ by active intervention.

In a step 530, the liquid starting component 210, 210′, 210″, 210″′, 210″″ is conveyed through the outlet 130, 130′, 130″, 130″′, 130″″ of the device 100, 100′, 100″, 100′″, 100″″. In a variant of step 530, the conveying is triggered solely by gravity. In a preferred variant of step 530, the conveying takes place through active intervention by the user of the device 100, 100′, 100″, 100″′, 100″″. For example, the user of the device 100, 100′, 100″, 100″′, 100″″ actuates a conveying means 170, 170″ such as a piston or a rubber balloon in order to convey the liquid starting component 210, 210′, 210″, 210″′, 210″″ through the outlet 130, 130′, 130″ of the device 100, 100′, 100″, 100″′, 100″″.

Preferably, the device 100, 100′, 100″, 100″′, 100″″, together with a mixing unit 410′, 410′, 410″, is part of a mixing device 400, 400′, 400″, so that the conveying in step 530 brings the liquid starting component 210, 210′, 210″, 210″′, 210″″ into the mixing unit 410′, 410′, 410″.

In an optional step 540, mixing of a bone cement paste takes place in the mixing unit 410′, 410′, 410″ of the mixing device 400′, 400′, 400″ from the liquid starting component 210, 210′, 210″, 210′″, 210″″ and a bone cement powder 215, 215′, 215″ stored in the mixing unit 410, 410′, 410″. For this purpose, the mixing unit 410, 410″ preferably has a mixing element 420, 420″, preferably in the form of a mixing rod.

In an optional step 550, the in the mixing device 400, 400′, 400″ is discharged directly from it at a desired location. For this purpose, the mixing device 400, 400′, 400″ preferably comprises a dispensing plunger which is movably mounted axially within the mixing unit 410, 410′, 410″ in order to dispense the provided bone cement paste from the mixing device 400, 400′, 400″, in particular from the mixing unit 410, 410′, 410″.

### REFERENCE SIGNS

100, 100′, 100″ 100′″, 100″″ Device
110, 110′, 110″ Housing
120, 120′, 120″ Holder
130, 130′, 130″ 130″′, 130″″ Outlet
140, 140′, 140″ Deformable region
145, 145′, 145″ Pressure piece
150, 150′, 150″ Filter element
160, 160′, 160″ Collection region
170, 170″ Conveying means
175 Cylinder
200. 200′, 200″ 200″′, 200″″ Glass ampoule
210, 210′, 210″ 210″′, 210″″ Liquid starting component
215, 215′, 215″ Bone cement powder
220, 220′, 220″ Ampoule head
230, 230′, 230″ Ampoule throat
240, 240′, 240″ Ampoule body
400, 400′, 400″ Mixing device
410, 410′, 410″ Mixing unit
420, 420″ Mixing element
430 Piston-cylinder system
440 Tube
450 Stand element
500 Method
510 Provision
520 Fluid-conducting opening
530 Conveying
540 Mixing
550 Dispensing

1. A device for dispensing a liquid starting component of a bone cement paste from a glass ampoule, comprising
a housing, a holder arranged in the housing for receiving a glass ampoule, an outlet for dispensing the liquid starting component from the housing,
wherein
the housing has a deformable region which is designed and arranged to be displaceable by pressure against the glass ampoule received in the holder and to open the glass ampoule in a fluid-conducting manner.

2. The device according to claim 1, wherein the deformable region of the housing has, on the side facing the holder, a substantially dimensionally stable pressure piece which can be displaced by pressure against the glass ampoule received in the holder.

3. The device according to claim 1, wherein the deformable region has a partial region, in particular a circumferential partial region, having a thinner wall thickness than the rest of the housing.

4. The device according to claim 1, wherein the deformable region is formed from an elastic material at least in portions.

5. The device according to claim 1, wherein the deformable region is arranged between the holder and the outlet.

6. The device according to claim 1, wherein the holder is designed to store a glass ampoule in the housing in such a way that an ampoule head of the glass ampoule adjoins the deformable region of the housing.

7. The device according to claim 1, wherein between the holder and the outlet, a filter element is arranged that is designed to retain, in the device, fragments of a glass ampoule opened in a fluid-conducting manner in the device.

8. The device according to claim 7, wherein the filter element is arranged between the deformable region and the outlet.

9. The device according to claim 1, wherein a collection region is arranged between the holder and the outlet in order to receive a liquid starting component from a glass ampoule opened in a fluid-conducting manner in the device.

10. The device according to claim 9, comprising a conveying means for conveying a liquid starting component from the collection region through the outlet out of the device.

11. The device according to claim 10, wherein the conveying means is a rubber balloon.

12. The device according to claim 10, wherein the conveying means is a piston.

13. A mixing device for providing a bone cement paste from two starting components, comprising
a device according to claim 1 and a mixing unit, wherein the mixing unit is designed and arranged to mix the bone cement paste from the two starting components.

14. The mixing device according to claim 13, wherein the device contains a glass ampoule comprising a liquid starting component as the first starting component of the bone cement paste, and the mixing unit contains a bone cement powder as the second starting component of the bone cement paste.

15. A method for dispensing a liquid starting component of a bone cement paste from a glass ampoule by means of a device according to claim 1, comprising the following steps:
a. providing a glass ampoule containing the liquid starting component in the holder;
b. opening the glass ampoule in a fluid-conducting manner by exerting pressure on the deformable region so that this region is pressed against the glass ampoule;
c. conveying the liquid starting component through the outlet out of the device.
